(19) United States
(12) Patent Application Publication
(10) Pub. No.: US 2023/0181472 A1
(43) Pub. Date: Jun. 15, 2023

(54) PROCESS OF MAKING IVABRADINE HYDROCHLORIDE DRUG PRODUCT

(71) Applicant: Amgen Inc., Thousand Oaks, CA (US)

(72) Inventors: Neil Obrad MACPHAIL, Newbury Park, CA (US); Angela Marie OLSOFSKY, Moorpark, CA (US); Tiffany Lee THIEL, Thousand Oaks, CA (US); Yong XIE, Thousand Oaks, CA (US)

(73) Assignee: AMGEN INC., Thousand Oaks, CA (US)

(21) Appl. No.: 17/283,068

(22) PCT Filed: Oct. 29, 2019
(86) PCT No.: PCT/US19/58433

§ 371 Date: Apr. 6, 2021

Related U.S. Application Data
(60) Provisional application No. 62/752,622, filed on Oct. 30, 2018.

(51) Int. Cl. A61K 9/16; A61K 31/55; A61K 9/20; A61K 9/28

(52) U.S. Cl. CPC A61K 9/1694; A61K 31/55; A61K 9/1623; A61K 9/1652; A61K 9/1617; A61K 9/1611; A61K 9/2018; A61K 9/2059; A61K 9/2013; A61K 9/2009; A61K 9/2095; A61K 9/2893; A61K 9/284

(57) ABSTRACT

A new process for making ivabradine hydrochloride drug product reduces the amount of amorphous ivabradine hydrochloride in the drug product.

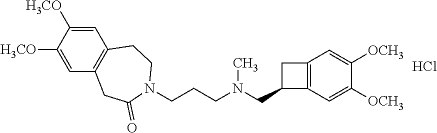 [SMILES: COc1cc2c(cc1OC)CC(=O)N(CCCN(C)C[C@H]1Cc3cc(OC)c(OC)cc31)CC2 ; Cl]

### CROSS REFERENCES TO RELATED APPLICATIONS

This application claims the benefit of U.S. Provisional Application No. 62/752,622, filed on Oct. 30, 2018, which is hereby incorporated by reference in its entirety and for all purposes as if fully set forth herein.

### FIELD OF THE INVENTION

The present invention relates to a novel process for making ivabradine hydrochloride drug product. The new process provides ivabradine hydrochloride drug product and material for making drug product that includes ivabradine hydrochloride as a mixture of monohydrate and tetrahydrate crystalline forms with a reduction in the amount of amorphous ivabradine hydrochloride.

### BACKGROUND OF THE INVENTION

Ivabradine hydrochloride has the structure shown below:

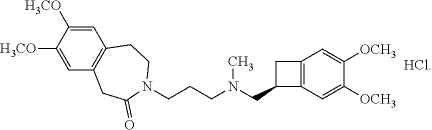 [SMILES: COc1cc2c(cc1OC)CC(=O)N(CCCN(C)C[C@H]1Cc3cc(OC)c(OC)cc31)CC2 ; "HCl."]

In 2015, the United States Food and Drug Administration approved ivabradine hydrochloride for reducing the risk of hospitalization for worsening heart failure in patients with stable, symptomatic chronic heart failure with left ventricular ejection fraction less than or equal to 35% who are in sinus rhythm with a resting heart rate of 70 or greater beats per minute and either are on maximally tolerated doses of beta-blockers or have a contraindication to beta-clocker use.

Ivabradine hydrochloride is currently available in the United States in 5 mg and 7.5 mg tablets. The ivabradine hydrochloride present in the 5 mg and 7.5 mg tablets is a mixture of monohydrate and tetrahydrate crystalline forms. The monohydrate crystalline form is referred to as the γ crystalline form and is described in U.S. Pat. Nos. 7,361,650 and 7,867,996. The tetrahydrate crystalline form is referred to as the β crystalline form and is described in U.S. Pat. Nos. 7,361,649 and 7,879,842.

The process used to make ivabradine hydrochloride drug product typically starts with the anhydrous ivabradine hydrochloride crystalline form referred to as the α crystalline form which is described in U.S. Pat. No. 7,867,994. During the process, the anhydrous α crystalline form of ivabradine hydrochloride is mixed with water and the other materials in the drug product. This process leads to the mixture of the γ monohydrate crystalline form and the β tetrahydrate crystalline form in the final drug product. This process may also result in small amounts of amorphous ivabradine hydrochloride being present in the final drug product. Reduction or elimination of amorphous ivabradine hydrochloride is desirable in the drug product to reduce oxidative degradation.

A need for an improved process for making ivabradine hydrochloride drug product exists which reduces the amount of amorphous ivabradine hydrochloride in the final drug product.

### SUMMARY OF THE INVENTION

In one aspect, the invention provides a method for producing ivabradine hydrochloride granule product. The method includes:
a) mixing lactose monohydrate, maize starch, maltodextrin, ivabradine hydrochloride, and water to provide a wet granule product, wherein the percent of lactose monohydrate by weight used to make the wet granule product ranges from 55% to 65% based on the total combined weight of the lactose monohydrate, maize starch, maltodextrin, ivabradine hydrochloride, and water;
the percent of maize starch by weight used to make the wet granule product ranges from 17% to 22% based on the total combined weight of the lactose monohydrate, maize starch, maltodextrin, ivabradine hydrochloride, and water;
the percent of maltodextrin by weight used to make the wet granule product ranges from 8% to 11% based on the total combined weight of the lactose monohydrate, maize starch, maltodextrin, ivabradine hydrochloride, and water;
the percent of ivabradine hydrochloride by weight used to make the wet granule product ranges from 4% to 8% based on the total combined weight of the lactose monohydrate, maize starch, maltodextrin, ivabradine hydrochloride, and water; and
the percent of water by weight used to make the wet granule product ranges from 5% to 8% based on the total combined weight of the lactose monohydrate, maize starch, maltodextrin, ivabradine hydrochloride, and water; and
b) drying the wet granule product to a level of less than or equal to 4% loss on drying to provide the ivabradine hydrochloride granule product,
wherein the wet granule product is dried in a fluid bed dryer with an inlet air temperature ranging from 37° C. to 44° C., with an inlet air dewpoint ranging from 7° C. to 12° C., and with an airflow ranging from 300 to 600 cubic feet per minute.

In another aspect, the invention provides a method of making ivabradine hydrochloride precompression product. The method includes:
a) milling the ivabradine hydrochloride granule product produced using the method of any one of the embodiments described herein to form a milled ivabradine granule product; and
b) mixing the milled ivabradine granule product with magnesium stearate and colloidal silica to produce the ivabradine hydrochloride precompression product, wherein
the percent of magnesium stearate by weight used to make the ivabradine hydrochloride precompression product ranges from 0.4% to 0.6% based on the total combined weight of the lactose monohydrate, maize starch, maltodextrin, ivabradine hydrochloride, magnesium stearate, and colloidal silica in the ivabradine hydrochloride precompression product; and
the percent of colloidal silica by weight used to make the ivabradine hydrochloride precompression product ranges from 0.1% to 0.3% based on the total combined weight of the lactose monohydrate, maize starch, maltodextrin, ivabradine hydrochloride, magnesium stearate, and colloidal silica in the ivabradine hydrochloride precompression product.

In another aspect, the invention provides a method for making precoated tablets of ivabradine hydrochloride. The method includes: compressing the ivabradine hydrochloride precompression product of any one of the embodiments to produce the precoated tablets of ivabradine hydrochloride.

In still another aspect, the invention provides a method of forming ivabradine hydrochloride drug product. The method includes:
a) coating the precoated tablets of ivabradine hydrochloride of any of the embodiments with an aqueous suspension or solution comprising a coloring agent to form a color-coated ivabradine hydrochloride tablet; and
b) coating the color-coated ivabradine hydrochloride tablet with an aqueous solution of a polishing agent.

Other objects, features and advantages of the invention will become apparent to those skilled in the art from the following description and claims.

### DETAILED DESCRIPTION OF THE INVENTION

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the standard deviation found in their respective testing measurements.

As used herein, if any variable occurs more than one time in a chemical formula, its definition on each occurrence is independent of its definition at every other occurrence. If the chemical structure and chemical name conflict, the chemical structure is determinative of the identity of the compound. The compounds of the present disclosure may contain one or more chiral centers and/or double bonds and therefore, may exist as stereoisomers, such as double-bond isomers (i.e., geometric isomers), enantiomers or diastereomers. Accordingly, any chemical structures within the scope of the specification depicted, in whole or in part, with a relative configuration encompass all possible enantiomers and stereoisomers of the illustrated compounds including the stereoisomerically pure form (e.g., geometrically pure, enantiomerically pure or diastereomerically pure) and enantiomeric and stereoisomeric mixtures. Enantiomeric and stereoisomeric mixtures can be resolved into the component enantiomers or stereoisomers using separation techniques or chiral synthesis techniques well known to the skilled artisan.

The term “comprising” is meant to be open ended, i.e., all encompassing and non-limiting. It may be used herein synonymously with “having” or “including”. Comprising is intended to include each and every indicated or recited component or element(s) while not excluding any other components or elements. For example, if a composition is said to comprise A and B. This means that the composition has A and B in it, but may also include C or even C, D, E, and other additional components.

“Pharmaceutically acceptable excipient” refers to a broad range of ingredients that may be combined with a compound or salt of the present invention to prepare a pharmaceutical composition or formulation. Typically, excipients include, but are not limited to, diluents, colorants, vehicles, anti-adherants, glidants, disintegrants, flavoring agents, coatings, binders, sweeteners, lubricants, sorbents, preservatives, and the like.

Reference will now be made in detail to embodiments of the present disclosure. While certain embodiments of the present disclosure will be described, it will be understood that it is not intended to limit the embodiments of the present disclosure to those described embodiments. To the contrary, reference to embodiments of the present disclosure is intended to cover alternatives, modifications, and equivalents as may be included within the spirit and scope of the embodiments of the present disclosure as defined by the appended claims.

### Embodiments

The embodiments listed below are presented in numbered form for convenience and for ease and clarity of reference in referring back to multiple embodiments.

In a first embodiment, the invention provides a method for producing ivabradine hydrochloride granule product. The method comprises:
a) mixing lactose monohydrate, maize starch, maltodextrin, ivabradine hydrochloride, and water to provide a wet granule product, wherein the percent of lactose monohydrate by weight used to make the wet granule product ranges from 55% to 65% based on the total combined weight of the lactose monohydrate, maize starch, maltodextrin, ivabradine hydrochloride, and water;
the percent of maize starch by weight used to make the wet granule product ranges from 17% to 22% based on the total combined weight of the lactose monohydrate, maize starch, maltodextrin, ivabradine hydrochloride, and water;
the percent of maltodextrin by weight used to make the wet granule product ranges from 8% to 11% based on the total combined weight of the lactose monohydrate, maize starch, maltodextrin, ivabradine hydrochloride, and water;
the percent of ivabradine hydrochloride by weight used to make the wet granule product ranges from 4% to 8% based on the total combined weight of the lactose monohydrate, maize starch, maltodextrin, ivabradine hydrochloride, and water; and
the percent of water by weight used to make the wet granule product ranges from 5% to 8% based on the total combined weight of the lactose monohydrate, maize starch, maltodextrin, ivabradine hydrochloride, and water; and
b) drying the wet granule product to a level of less than or equal to 4% loss on drying to provide the ivabradine hydrochloride granule product,
wherein the wet granule product is dried in a fluid bed dryer with an inlet air temperature ranging from 37° C. to 44° C., with an inlet air dewpoint ranging from 7° C. to 12° C., and with an airflow ranging from 300 to 600 cubic feet per minute.

2. The method of embodiment 1, wherein
the percent of lactose monohydrate by weight used to make the wet granule product ranges from 59% to 62% based on the total combined weight of the lactose monohydrate, maize starch, maltodextrin, ivabradine hydrochloride, and water;
the percent of maize starch by weight used to make the wet granule product ranges from 18% to 20% based on the total combined weight of the lactose monohydrate, maize starch, maltodextrin, ivabradine hydrochloride, and water;
the percent of maltodextrin by weight used to make the wet granule product ranges from 9% to 11% based on the total combined weight of the lactose monohydrate, maize starch, maltodextrin, ivabradine hydrochloride, and water;
the percent of ivabradine hydrochloride by weight used to make the wet granule product ranges from 5% to 6% based on the total combined weight of the lactose monohydrate, maize starch, maltodextrin, ivabradine hydrochloride, and water; and
the percent of water by weight used to make the wet granule product ranges from 5.2% to 6.5% based on the total combined weight of the lactose monohydrate, maize starch, maltodextrin, ivabradine hydrochloride, and water.

3. The method of embodiment 1, wherein
the percent of lactose monohydrate by weight used to make the wet granule product ranges from 60% to 62% based on the total combined weight of the lactose monohydrate, maize starch, maltodextrin, ivabradine hydrochloride, and water;
the percent of maize starch by weight used to make the wet granule product ranges from 19% to 20% based on the total combined weight of the lactose monohydrate, maize starch, maltodextrin, ivabradine hydrochloride, and water;
the percent of maltodextrin by weight used to make the wet granule product ranges from 9% to 10% based on the total combined weight of the lactose monohydrate, maize starch, maltodextrin, ivabradine hydrochloride, and water;
the percent of ivabradine hydrochloride by weight used to make the wet granule product ranges from 5% to 5.5% based on the total combined weight of the lactose monohydrate, maize starch, maltodextrin, ivabradine hydrochloride, and water; and
the percent of water by weight used to make the wet granule product ranges from 5.2% to 5.5% based on the total combined weight of the lactose monohydrate, maize starch, maltodextrin, ivabradine hydrochloride, and water.

4. The method of embodiment 1, wherein
the percent of lactose monohydrate by weight used to make the wet granule product ranges from 60.9% to 61.0% based on the total combined weight of the lactose monohydrate, maize starch, maltodextrin, ivabradine hydrochloride, and water;
the percent of maize starch by weight used to make the wet granule product ranges from 19.05% to 19.1% based on the total combined weight of the lactose monohydrate, maize starch, maltodextrin, ivabradine hydrochloride, and water;
the percent of maltodextrin by weight used to make the wet granule product ranges from 9.5% to 9.6% based on the total combined weight of the lactose monohydrate, maize starch, maltodextrin, ivabradine hydrochloride, and water;
the percent of ivabradine hydrochloride by weight used to make the wet granule product ranges from 5.1% to 5.2% based on the total combined weight of the lactose monohydrate, maize starch, maltodextrin, ivabradine hydrochloride, and water; and
the percent of water by weight used to make the wet granule product ranges from 5.2% to 5.4% based on the total combined weight of the lactose monohydrate, maize starch, maltodextrin, ivabradine hydrochloride, and water.

5. The method of embodiment 1, wherein
the amount of lactose monohydrate used to make the wet granule product ranges from 63 kg to 65 kg;
the amount of maize starch used to make the wet granule product ranges from 19 kg to 21 kg;
the amount of maltodextrin used to make the wet granule product ranges from 9.5 kg to 10.5 kg;
the amount of ivabradine hydrochloride used to make the wet granule product ranges from 5.2 kg to 5.5 kg; and
the amount of water used to make the wet granule product ranges from 5.3 kg to 6.0 kg.

6. The method of embodiment 1, wherein
the amount of lactose monohydrate used to make the wet granule product ranges from 63.8 kg to 64 kg;
the amount of maize starch used to make the wet granule product ranges from 19.5 kg to 20.5 kg;
the amount of maltodextrin used to make the wet granule product ranges from 9.8 kg to 10.2 kg;
the amount of ivabradine hydrochloride used to make the wet granule product ranges from 5.3 kg to 5.45 kg; and
the amount of water used to make the wet granule product ranges from 5.4 kg to 5.8 kg.

7. The method of embodiment 1, wherein
the amount of lactose monohydrate used to make the wet granule product ranges from 63.9 kg to 63.92 kg;
the amount of maize starch used to make the wet granule product ranges from 19.9 kg to 20.1 kg;
the amount of maltodextrin used to make the wet granule product ranges from 9.9 kg to 10.1 kg;
the amount of ivabradine hydrochloride used to make the wet granule product ranges from 5.39 kg to 5.41 kg; and
the amount of water used to make the wet granule product ranges from 5.48 kg to 5.52 kg.

8. The method of embodiment 1, wherein
the amount of lactose monohydrate used to make the wet granule product is 63.91 kg;
the amount of maize starch used to make the wet granule product is 20.0 kg;
the amount of maltodextrin used to make the wet granule product is 10.0 kg;
the amount of ivabradine hydrochloride used to make the wet granule product is 5.39 kg; and
the amount of water used to make the wet granule product is 5.5 kg.

9. The method of embodiment 1, wherein
the percent of lactose monohydrate by weight used to make the wet granule product ranges from 56% to 59% based on the total combined weight of the lactose monohydrate, maize starch, maltodextrin, ivabradine hydrochloride, and water;
the percent of maize starch by weight used to make the wet granule product ranges from 17% to 20% based on the total combined weight of the lactose monohydrate, maize starch, maltodextrin, ivabradine hydrochloride, and water;
the percent of maltodextrin by weight used to make the wet granule product ranges from 9% to 11% based on the total combined weight of the lactose monohydrate, maize starch, maltodextrin, ivabradine hydrochloride, and water;
the percent of ivabradine hydrochloride by weight used to make the wet granule product ranges from 7% to 8% based on the total combined weight of the lactose monohydrate, maize starch, maltodextrin, ivabradine hydrochloride, and water; and
the percent of water by weight used to make the wet granule product ranges from 6% to 8% based on the total combined weight of the lactose monohydrate, maize starch, maltodextrin, ivabradine hydrochloride, and water.

10. The method of embodiment 1, wherein
the percent of lactose monohydrate by weight used to make the wet granule product ranges from 57% to 59% based on the total combined weight of the lactose monohydrate, maize starch, maltodextrin, ivabradine hydrochloride, and water;
the percent of maize starch by weight used to make the wet granule product ranges from 18% to 20% based on the total combined weight of the lactose monohydrate, maize starch, maltodextrin, ivabradine hydrochloride, and water;
the percent of maltodextrin by weight used to make the wet granule product ranges from 9% to 10% based on the total combined weight of the lactose monohydrate, maize starch, maltodextrin, ivabradine hydrochloride, and water;
the percent of ivabradine hydrochloride by weight used to make the wet granule product ranges from 7% to 8% based on the total combined weight of the lactose monohydrate, maize starch, maltodextrin, ivabradine hydrochloride, and water; and
the percent of water by weight used to make the wet granule product ranges from 6% to 7% based on the total combined weight of the lactose monohydrate, maize starch, maltodextrin, ivabradine hydrochloride, and water.

11. The method of embodiment 1, wherein
the percent of lactose monohydrate by weight used to make the wet granule product ranges from 57.5% to 58% based on the total combined weight of the lactose monohydrate, maize starch, maltodextrin, ivabradine hydrochloride, and water;
the percent of maize starch by weight used to make the wet granule product ranges from 18.5% to 19% based on the total combined weight of the lactose monohydrate, maize starch, maltodextrin, ivabradine hydrochloride, and water;
the percent of maltodextrin by weight used to make the wet granule product ranges from 9.3% to 9.6% based on the total combined weight of the lactose monohydrate, maize starch, maltodextrin, ivabradine hydrochloride, and water;
the percent of ivabradine hydrochloride by weight used to make the wet granule product ranges from 7.5% to 7.8% based on the total combined weight of the lactose monohydrate, maize starch, maltodextrin, ivabradine hydrochloride, and water; and
the percent of water by weight used to make the wet granule product ranges from 6% to 6.5% based on the total combined weight of the lactose monohydrate, maize starch, maltodextrin, ivabradine hydrochloride, and water.

12. The method of embodiment 1, wherein
the amount of lactose monohydrate used to make the wet granule product ranges from 60 kg to 63 kg;
the amount of maize starch used to make the wet granule product ranges from 19 kg to 21 kg;
the amount of maltodextrin used to make the wet granule product ranges from 9.5 kg to 10.5 kg;
the amount of ivabradine hydrochloride used to make the wet granule product ranges from 7.5 kg to 8.5 kg; and
the amount of water used to make the wet granule product ranges from 6.0 kg to 6.9 kg.

13. The method of embodiment 1, wherein
the amount of lactose monohydrate used to make the wet granule product ranges from 60.8 kg to 62 kg;
the amount of maize starch used to make the wet granule product ranges from 19.5 kg to 20.5 kg;
the amount of maltodextrin used to make the wet granule product ranges from 9.8 kg to 10.4 kg;
the amount of ivabradine hydrochloride used to make the wet granule product ranges from 7.8 kg to 8.2 kg; and
the amount of water used to make the wet granule product ranges from 6.2 kg to 6.7 kg.

14. The method of embodiment 1, wherein
the amount of lactose monohydrate used to make the wet granule product ranges from 61.1 kg to 61.3 kg;
the amount of maize starch used to make the wet granule product ranges from 19.9 kg to 20.1 kg;
the amount of maltodextrin used to make the wet granule product ranges from 9.9 kg to 10.1 kg;
the amount of ivabradine hydrochloride used to make the wet granule product ranges from 8.0 kg to 8.2 kg; and
the amount of water used to make the wet granule product ranges from 6.4 kg to 6.6 kg.

15. The method of embodiment t1, wherein
the amount of lactose monohydrate used to make the wet granule is 61.22 kg;
the amount of maize starch used to make the wet granule product is 20.0 kg;
the amount of maltodextrin used to make the wet granule product is 10.0 kg;
the amount of ivabradine hydrochloride used to make the wet granule product is 8.9 kg; and
the amount of water used to make the wet granule product is 6.5 kg.

16. The method of any one of embodiments 1-15, wherein the wet granule product is dried in a fluid bed dryer with an inlet air temperature ranging from 39° C. to 43.2° C., with an inlet air dewpoint ranging from 9° C. to 11° C., and with an airflow ranging from 450 to 550 cubic feet per minute.

17. The method of any one of embodiments 1-15, wherein the wet granule product is dried in a fluid bed dryer with an inlet air temperature ranging from 40° C. to 43° C., with an inlet air dewpoint ranging from 9.5° C. to 10.5° C., and with an airflow ranging from 480 to 520 cubic feet per minute.

18. The method of any one of embodiments 1-15, wherein the wet granule product is dried in a fluid bed dryer with an inlet air temperature ranging from 40° C. to 43° C., with an inlet air dewpoint of 10° C., and with an airflow of 500 cubic feet per minute.

19. The method of any one of claims 1-18, wherein the method further comprises:
c) milling the wet granule product prior to drying the wet granule product.

20. The method of claim 19, wherein the milling of the wet granule product is conducted while maintaining the dew point at a range from 8° C. to 15° C., and maintaining an air inlet temperature of 8° C. to 15°.

21. The method of claim 19, wherein the milling of the wet granule product is conducted while maintaining the dew point at a range from 9° C. to 11° C., and maintaining an air inlet temperature of 9° C. to 11° C.

22. The method of claim 19, wherein the the milling of the wet granule product is conducted while maintaining the dew point at 10° C., and maintaining an air inlet temperature of 10° C.

23. A method of making ivabradine hydrochloride precompression product, the method comprising:
a) milling the ivabradine hydrochloride granule product produced using the method of any one of embodiments 1-22 to form a milled ivabradine granule product; and
b) mixing the milled ivabradine granule product with magnesium stearate and colloidal silica to produce the ivabradine hydrochloride precompression product, wherein
the percent of magnesium stearate by weight used to make the ivabradine hydrochloride precompression product ranges from 0.4% to 0.6% based on the total combined weight of the lactose monohydrate, maize starch, maltodextrin, ivabradine hydrochloride, magnesium stearate, and colloidal silica in the ivabradine hydrochloride precompression product; and
the percent of colloidal silica by weight used to make the ivabradine hydrochloride precompression product ranges from 0.1% to 0.3% based on the total combined weight of the lactose monohydrate, maize starch, maltodextrin, ivabradine hydrochloride, magnesium stearate, and colloidal silica in the ivabradine hydrochloride precompression product.

24. The method of embodiment 23, wherein
the percent of magnesium stearate by weight used to make the ivabradine hydrochloride precompression product ranges from 0.45% to 0.55% based on the total combined weight of the lactose monohydrate, maize starch, maltodextrin, ivabradine hydrochloride, magnesium stearate, and colloidal silica in the ivabradine hydrochloride precompression product; and
the percent of colloidal silica by weight used to make the ivabradine hydrochloride precompression product ranges from 0.15% to 0.25% based on the total combined weight of the lactose monohydrate, maize starch, maltodextrin, ivabradine hydrochloride, magnesium stearate, and colloidal silica in the ivabradine hydrochloride precompression product.

25. The method of embodiment 23, wherein
the percent of magnesium stearate by weight used to make the ivabradine hydrochloride precompression product ranges from 0.48% to 0.52% based on the total combined weight of the lactose monohydrate, maize starch, maltodextrin, ivabradine hydrochloride, magnesium stearate, and colloidal silica in the ivabradine hydrochloride precompression product;
the percent of colloidal silica by weight used to make the ivabradine hydrochloride precompression product ranges from 0.18% to 0.22% based on the total combined weight of the lactose monohydrate, maize starch, maltodextrin, ivabradine hydrochloride, magnesium stearate, and colloidal silica in the ivabradine hydrochloride precompression product.

26. The method of embodiment 23, wherein
the amount of magnesium stearate used to make the ivabradine hydrochloride precompression product ranges from 0.48 kg to 0.52 kg; and
the amount of colloidal silica used to make the ivabradine hydrochloride precompression product ranges from 0.18 kg to 0.22 kg.

27. The method of embodiment 23, wherein
the amount of magnesium stearate used to make the ivabradine hydrochloride precompression product is 0.50 kg; and
the amount of colloidal silica used to make the ivabradine hydrochloride precompression product is 0.20 kg.

28. A method for making precoated tablets of ivabradine hydrochloride, the method comprising: compressing the ivabradine hydrochloride precompression product of any one of embodiments 23-27 to produce the precoated tablets of ivabradine hydrochloride.

29. The method of embodiment 28, wherein the 100 mg of the ivabradine hydrochloride precompression product is compressed to form the precoated tablets of ivabradine hydrochloride.

30. A method of forming ivabradine hydrochloride drug product, the method comprising:
a) coating the precoated tablets of ivabradine hydrochloride of embodiment 28 or embodiment 29 with an aqueous suspension or solution comprising a coloring agent to form a color-coated ivabradine hydrochloride tablet; and
b) coating the color-coated ivabradine hydrochloride tablet with an aqueous solution of a polishing agent.

31. The method of embodiment 30, wherein the coloring agent is Sepifilm™ salmon film-coating agent.

32. The method of embodiment 30 or embodiment 31, wherein the polishing agent is polyethylene glycol 6000.

The invention is further described by reference to the following examples, which are intended to exemplify the claimed invention but not to limit it in any way.

### EXAMPLES

### Example 1.0

### Preparation of Ivabradine Hydrochloride Drug Product

Two drug product tablet strengths (5 mg and 7.5 mg) of ivabradine hydrochloride were manufactured using wet granulation technology. The wet granulation technology includes the following steps:
(1) high shear wet granulation;
(2) wet milling;
(3) fluid bed drying;
(4) dry milling;
(5) blending;
(6) compression; and
(7) film coating.

The manufacturing process is depicted in Schemes 1-3 and further details are provided below. The amounts used to prepare the 5 mg and 7.5 mg tablets are presented in Table 1 and Table 2. The basic equipment used in the process is described below. A Diosna CGS 300 granulation train was used to perform the key process steps for the granulation process.
High shear wet granulator: Diosna 300 L high shear granulator
Inline Mill: Frewitt Coniwitt 200 in-line mill
Fluid bed dryer: Diosna CAP 300 fluid bed dryer
Compressor: Korsch XL 200 tablet press
Film Coating Pan: Accela-Cota 48″ coater

Dispensing

The core tablet ingredients of lactose monohydrate, ivabradine hydrochloride, maltodextrin, and maize starch were dispensed and transferred into a 160 L intermediate bulk container.

The coating ingredients of polyethylene glycol 6000 and Sepifilm™ 4219 salmon film-coating agent were dispensed into individual containers.

Granulation

The wet granulation process was conducted in a 300 L high shear wet granulator with water serving as the granulating fluid.

The mixture in the intermediate bulk container containing the lactose monohydrate, ivabradine hydrochloride, maltodextrin, and maize starch was charged to the high shear granulator and the ingredients were dry mixed to achieve a homogeneous blend. Next, the granulating fluid (water) was added. After addition of the water was complete, the impeller speed was increased and wet massing occurred. The process parameters and in-process controls are provided in Scheme 1 (Wet Granulation).

Wet Milling/Transfer

Upon completion of the granulations process, the granules obtained were wet milled using an inline mill and transferred into a fluid bed dryer using cool air. The process parameters and in-process controls are provided in Scheme 1 (Wet Milling/Transfer). For convenience, the granule product obtained prior to the drying step will be referred to as the “wet granule product”. This term will apply to material both before and after milling.

Drying

After the transfer process was complete, the granules were dried until loss on drying of less than or equal to 3.5% was achieved. The process parameters and in-process controls are provided in Scheme 2 (Drying). For convenience, the granule product, dried as described above, prior to addition of the magnesium stearate and colloidal silica will be referred to as the “granule product”. Notably, the dried granule product includes some water as noted above and includes the monohydrate and tetrahydrate crystalline forms of the ivabradine hydrochloride.

Milling

After drying, the granules were conveyed through an inline conical mill to a 300 L intermediate bulk container. The yield of the dry milled granules was measured. If the process yield was greater than or equal to 95.0%, no adjustment was made to the pre-weighed extra-granular excipients. If the accountable yield was less than 95.0%, the extragranular excipients were adjusted to reflect the dry milled granulate yield. Next, magnesium stearate and colloidal silica were passed through the conical mill into the intermediate bulk container. The process parameters and in-process controls are provided in Scheme 2 (Milling) For convenience, the milled granule product, dried and milled as described above, prior to addition of the magnesium stearate and colloidal silica will be referred to as the “milled dried granule product”. Notably, the milled dried granule product includes some water and includes the monohydrate and tetrahydrate crystalline forms of the ivabradine hydrochloride.

Lubrication

The milled granule, magnesium stearate and colloidal silica were blended in the intermediate bulk container. The magnesium stearate was screened through a #40 mesh before dispensing and the colloidal silica was screened through a #20 mesh before dispensing. The process parameters and in-process controls are provided in Scheme 2 (Lubrication). For convenience, the product produced after mixing of the milled dried granule product with the magnesium stearate and colloidal silica will be referred to as the “precompression product”.

Compression

Tablets were then compressed to a target mass of 100 mg for both strengths. The 7.5 mg tablet was compressed using a triangular tooling, and the 5 mg tablet was compressed using an oval shaped tooling. Throughout the compression operation, individual tablet weight, the average weight of 10 tablets, and tablet hardness and thickness were monitored at predetermined intervals. The process parameters are provided in Scheme 3 (Compression). For convenience, the product produced after compression will be referred to as the “precoated tablet”.

Film Coating

The color-coating suspension and polishing solutions were prepared in separate stirred tanks by slowly adding either the Sepifilm™ salmon film-coating agent or polyethylene glycol 6000 into purified water while mixing until uniform. The color-coating suspension was de-aerated by a reduction of mixing speed prior to use. The core tablets were then transferred to a film coating pan for the application of the Sepifilm™ salmon color-coating suspension. The tablets were coated with a fixed quantity of suspension followed by a polishing step where a fixed quantity of polyethylene 6000 solution was applied. The tablets were then cooled and samples were removed from the coating for determination of average tablet weight, acceptable quality limit, final drug product release testing, and reserves. The process parameters are provided in Scheme 3 (Film Coating). The final polished tablets were then transferred from the coating pan directly into appropriate bulk containers and double lined with polyethylene bags, and a desiccant pouch was placed between the inner and outer bag. For convenience, the product produced after coating with the Sepifilm™ salmon and polyethylene 6000 will be referred to as the “drug product”.

TABLE 1
Ivabradine 5 mg Tablet Composition

| | | Composition | Mass |
|---|---|---|---|
| Core Tablet | Functionality | (w/w %) | (kg) |
| Ivabradine hydrochloride¹ | Drug Substance | 5.39 | 5.39 |
| Lactose monohydrate | Diluent | 63.91 | 63.91 |
| Maize starch | Disintegrant | 20.00 | 20.00 |
| Maltodextrin | Binder | 10.00 | 10.00 |
| Magnesium stearate | Lubricant | 0.50 | 0.50 |
| Colloidal silicon dioxide | Glidant | 0.20 | 0.20 |
| Purified water² | Granulating Fluid | — | 5.5 |
| Core Table Total | — | 100.0 | 100.0 |

Film Coating

| | | | |
|---|---|---|---|
| Sepifilm ™ 4219, Salmon³ | Color Coating material | 1.942 | 5.90 |
| Purified Water³ | Coating solvent | — | 58.16 |
| Polyethylene glycol 6000⁴ | Smoothing agent | 0.0578 | 2.0 |
| Purified Water³ | Coating solvent | — | 18.0 |
| Film Coated Tablet Total | — | 102.0 | 102.0 |

¹The molecular weights of ivabradine HCl and ivabradine free base are 505.1 g/mol and 468.6 g/mole respectively. The free base accounts for 92.77% of the salt. The starting material used was the anhydrous a crystalline form of ivabradine hydrochloride.
²Water was removed during the manufacture of the drug product. However, the drug substance in the drug product was predominantly a mixture of the monohydrate γ crystalline form and tetrahydrate β crystalline form of ivabradine hydrochloride.
³A maximum of 5.90 kg was used per lot prepared as a 9.2% w/w suspension. The theoretical quantity based on 100 kg batch size is 1.942 kg.
⁴A maximum of 2.0 kg was used per lot prepared as a 10.0% w/w solution. The theoretical quantity based on 100 kg batch size is 0.0578 kg.

TABLE 2
Ivabradine 7.5 mg Tablet Composition

| | | Composition | Mass |
|---|---|---|---|
| Core Tablet | Functionality | (w/w %) | (kg) |
| Ivabradine hydrochloride¹ | Drug Substance | 8.09 | 8.09 |
| Lactose monohydrate | Diluent | 61.22 | 61.22 |
| Maize starch | Disintegrant | 20.00 | 20.00 |
| Maltodextrin | Binder | 10.00 | 10.00 |
| Magnesium stearate | Lubricant | 0.50 | 0.50 |
| Colloidal silicon dioxide | Glidant | 0.20 | 0.20 |
| Purified water² | Granulating Fluid | — | 6.5 |
| Core Table Total | — | 100.0 | 100.0 |

Film Coating

| | | | |
|---|---|---|---|
| Sepifilm ™ 4219, Salmon³ | Color Coating material | 1.942 | 5.90 |
| Purified Water³ | Coating solvent | — | 58.16 |
| Polyethylene Glycol 6000⁴ | Smoothing agent | 0.0578 | 2.0 |
| Purified Water³ | Coating solvent | — | 18.0 |
| Film Coated Tablet Total | — | 102.0 | 102.0 |

¹The molecular weights of ivabradine HCl and ivabradine free base are 505.1 g/mol and 468.6 g/mole respectively. The free base accounts for 92.77% of the salt. The starting material used was the anhydrous a crystalline form of ivabradine hydrochloride.
²Water was removed during the manufacture of the drug product. However, the drug substance in the drug product was predominantly a mixture of the monohydrate γ crystalline form and tetrahydrate β crystalline form of ivabradine hydrochloride.
³A maximum of 5.90 kg was used per lot prepared as a 9.2% w/w suspension. The theoretical quantity based on 100 kg batch size is 1.942 kg.
⁴A maximum of 2.0 kg was used per lot prepared as a 10.0% w/w solution. The theoretical quantity based on 100 kg batch size is 0.0578 kg.

All the samples tested from process characterization lots of ivabradine hydrochloride drug product for the 5 mg and 7.5 mg tablet strengths met the target specifications. The results of the tests and the adjustments to the process parameters within the granulation, wet mill transfer and drying steps produced the desired stability endpoints. The process includes an increase in granulation fluid addition from 5% to 5.5% w/w and 5% to 6% for both 5 mg and 7.5 mg granulations respectively.

The wet mill transfer used the same air flow 1250 cfm, but the inlet air dew point was increased to 10° C. from 5° C. and the air temperature reduced from 30° C. to 10° C., minimizing drying during the transfer from the granulator to the fluid bed dryer.

During drying, the airflow was maintained at 500 cfm, but the inlet air temperature was reduced from 50° C. to 40° C. and the inlet air dew point was increased to 10° C. from 5° C. to reduce the overall drying rate.

Characterization of Drug Substance in Ivabradine Hydrochloride Drug Product

During characterization of the ivabradine hydrochloride chrystalline forms, Fourier Transform Raman Spectroscopy was used to evaluate different peak shapes. A Thermo Scientific Nicolet NXR FT-Raman equipped with liquid nitrogen cooled germanium (Ge) detector (Thermo Scientific) was used for FT-Raman signal collection. Peak broadening was most defined at a Raman shift of 705 cm⁻¹. The peak heights were normalized at 705 cm⁻¹ and the width of the peak was characterized as full width at half maximum (FWHM). Table 3 highlights the FWHM of pure tetrahydrate and amorphous drug substance with an approximate doubling of peak width for purely amorphous ivabradine hydrochloride. In the formulated product, a shift in FWHM of 3 cm⁻¹ (9.6 to 12.6 cm⁻¹) provided enough amorphous content in the drug product to fail accelerated stability testing. Modifying the process conditions as described herein produced FWHM values of 10.3 and 9.6 cm⁻¹ for the 5 mg and 7.5 mg drug products respectively showing the significant reduction in the amount of amorphous ivabradine hydrochloride present in the drug product.

TABLE 3
Ivabradine 7.5 mg Tablet Composition

| | FWHM |
|---|---|
| | (cm⁻¹) of |
| | 705 cm⁻¹ |
| Description | peak |
| Ivabradine hydrochloride tetrahydrate (pure) | 8.4 |
| Ivabradine hydrochloride amorphous (pure) | 16.3 |
| Formulated drug product (7.5 mg) with | 12.6 |
| failing stability performance | |
| Final Formulated drug product (5 mg) | 10.3 |
| characterized | |
| Final Formulated drug product (7.5 mg) | 9.6 |
| characterized | |

### Comparative Example

### Preparation of Ivabradine Hydrochloride Drug Product

The following description provides a general description of a process for making ivabradine hydrochloride drug product. The manufacturing process is depicted in Scheme 4 and further details are provided below.

Lactose monohydrate, maize starch and maltodextrin were dispensed and transferred to the granulation area. The ivabradine hydrochloride was received as pre-weighed bags of 10.78 kg. Magnesium stearate, colloidal silica, Sepifilm™ 4219 salmon film-coating agent and polyethylene glycol 6000 were dispensed into separate containers for use later in production.

The contents of the tote of excipients and ivabradine hydrochloride was added to a high shear granulator where the jacket temperature was controlled to heat up the materials. The materials were dry mixed to achieve a homogenous blend of ingredients and to reach a product temperature of greater than or equal to 45° C. Once this temperature was achieved, water was added over a period of 5 minutes. After the water addition was complete, the impeller speed was increased and wet massing occurred for 7.5 minutes. When the granulation was complete, a vacuum was pulled to dry the granules. This continued until a target LOD of less than or equal to 3.5% was achieved. Once the granules were dry, the material was passed through a conical mill into a 42 cubic foottote where, upon completion, the magnesium stearate and colloidal silica were also de-lumped through the mill into the tote.

The contents in the tote were blended to achieve adequate dispersion. The tote was then transferred above a rotary tablet compression machine. Tablets were compressed to a target mass of 100 mg for both doses but differentiated by shape. The 7.5 mg image was triangular and the 5 mg was an oval shape with a score to aid with tablet splitting. Target tablet hardness of 6 kP was used for both the 7.5 mg and 5 mg tablets.

On completion of the compression step, the tablets were transferred to a film coating unit for the application of an aqueous based suspension of Sepifilm™ salmon film-coating agent. The tablets were coated with a fixed quantity of the suspension followed by a polishing step using polyethylene glycol 6000. The final coated tablets were removed from the coating pan and placed directly into appropriate bulk containers.

In the following section, the process description is broken into the four separate unit operations including:
(1) granulation;
(2) lubrication;
(3) compression; and
(4) film coating.

The overall schematic of the drug product process and illustrated process steps are provided in Scheme 4. A single batch size of 400 kg was manufactured for both the 5 mg and 7.5 mg doses.

Granulation

Dispensing

The lactose monohydrate, maltodextrin and maize starch were charged into a tote. Ivabradine hydrochloride was received in a pre-weighed bag of 10.78 kg. Magnesium stearate and colloidal silica were dispensed into separate containers for use after granulation and drying.

Purified water (12 kg) was dispensed as the granulation fluid and added to a pressure pot.

Equipment Set-Up

The spray rate of the water was adjusted to achieve a rate of approximately 2.4 kg/minute. The cone mill was set up with a 1.27 mm grated screen for de-lumping the granule post cooling. The granulator jacket and lid were preheated to a temperature of 50° C.

Wet Granulation

Under a slight vacuum, approximately half of the tote containing the lactose monohydrate, maltodextrin, and maize starch were added to the granulator. The pre-dispensed bags of ivabradine hydrochloride wee then added to the granulator. The remaining lactose monohydrate, maltodextrin, and maize starch from the tote were then added to the granulator.

The temperature of the granulator jacket was then increased to 60° C. At this temperature, the ingredients were mixed for 10 minutes at an impeller speed of 55 revolutions per minute. After the dry mixing, if the temperature was less than 45° C., the mixture was heated with stirring at 3 revolutions per minute until a temperature of 45° C. or greater was achieved. Once the product temperature reached the set-point, water was spayed into the granulator at a granulator impeller speed of 55 revolutions per minute and at a slow chopper speed setting. Once the water had been added, the addition time was recorded and wet massing of the granule was started at an impeller speed of 80 revolutions per minute with a high chopper speed setting. The granule was wet massed for a target of 7 minutes and 30 seconds.

Drying

Once the granulation was complete, the jacket and lid temperatures were checked to see if they were still 60° C. The granulator was then placed under vacuum and the mixing speed was set to 12 revolutions per minute to promote drying of the granules. As the granule dries, the impeller speed was increased intermittently to promote uniform drying. The drying process was stopped once a loss on drying of less than or equal to 3.5% was achieved.

Cooling

After the loss on drying of the granule had dropped below 3.5%, the granule was cooled prior to discharge and milling. During cooling, the granule was continuously moving at a low impeller speed. Cooling of the granule started at a jacket and lid temperature of 45° C. and was incrementally decreased throughout until a product temperature of less than or equal to 30° C. was achieved.

Milling

A 42 cubic foot tote was positioned under the mill at the discharge of the granulator. The mill impeller speed was set to 1000 revolutions per minute and the granule was slowly discharged by opening the discharge valve and running the granulator impeller at a speed of 0.3 revolutions per minute for 15 minutes. After 15 minutes, the granulator impeller speed was increased to 0.5 revolutions per minute. The remaining granule was then discharged at a granulator impeller speed of 15 revolutions per minute. The weight of the milled granule was then obtained.

Lubricant Screening

The magnesium stearate and colloidal silica were added to a hopper positioned above the mill. The mill impeller speed was set to 1000 revolutions per minute and then the magnesium stearate and colloidal silica were added. The combined materials were transferred to the tote and the mass of the combined materials was obtained. The tote was then transferred to the blending area. The overall granulation control strategy is shown in Table 4.

TABLE 4
Ivabradine Granulation Control Strategy

| | | Parameter Target | Material |
|---|---|---|---|
| Unit Operation | In Process Control | Set-Point | Attribute |
| Dry Mixing | Temperature of heated | 60° C. | N/A |
| | jacket | | |
| | Impeller Speed | 55 RPM | |
| | Mixing time | 0 minutes | |
| Heating | Temperature of heated | 60° C. | Product |
| | jacket | | temperature |
| | Speed of mixer | 3 RPM | ≥45° C. |
| Granulation | Temperature of heated | 60° C. | N/A |
| | jacket | | |
| | Mass of water | 12 kg | |
| | Spraying time | 5 minutes | |
| | Impeller Speed | 55 RPM | |
| Wet Massing | Temperature of heated | 60° C. | N/A |
| | jacket | | |
| | Impeller speed | 80 RPM | |
| | Mixing time | 7.5 minutes | |
| Drying | Temperature of heated | 60° C. | Loss on |
| | jacket | | Drying |
| | Vacuum | 40 mBar | ≤3.5% w/w |
| | Impeller speed | Variable | |
| Cooling | Temperature of heated | 15° C. to 45° C. | Product |
| | jacket | | temperature |
| | Impeller speed | Variable | ≤30° C. |
| Discharging/ | Mill screen size | 1.27 mm grated | N/A |
| Milling | Mill Speed | 1000 RPM | |
| | Impeller Speed | 0.3 to 15 RPM | |
| Lubricant and | Mill screen size | 1.27 mm grated | N/A |
| glidant addition | Mill speed | 1000 RPM | |

Lubrication

The tote was positioned onto a tote blender and the speed was set to 7 revolutions per minute with a blending time of 5 minutes. The control strategy for the lubrication process is set forth in Table 5.

TABLE 5
Ivabradine Lubrication Control Strategy

| Unit | In Process | Parameter Target | |
|---|---|---|---|
| Operation | Control | Set-Point | Material Attribute |
| Diffusion | Fill Volume | ≥40% | Blend uniformity |
| Blending | Rotational speed | 7 RPM | RSD ≤ 5.0% |
| | Number of | 35 revolutions | No individual ± |
| | revolutions | | 10% of mean assay |

Compression

After lubrication, tablets were formed by compression on a rotary press as described in Tables 6 and 7.

TABLE 6
Ivabradine Tablet Images

| Ivabradine Potency | 5 mg | 7.5 mg |
|---|---|---|
| Shape | Oval | Triangle |
| Size | 8.5 mm × | 6.5 mm × |
| | 4.5 mm | 7.0 mm |
| Upper Face | Bisect | ″7.5″ |
| Lower Face | ″5″ | Plain |

TABLE 7
Ivabradine Compression Control Strategy

| Unit | In Process | Parameter Target | |
|---|---|---|---|
| Operation | Control | Set-Point | Material Attribute |
| Compression | Turret Speed | Within Validated | Weight Uniformity |
| | | Range | 95% to 105% |
| | | | target |
| | Feeder speed | Adjust to achieve | Average |
| | | consistent tablet | Hardness |
| | | weight | 5 mg: 3-9 kP |
| | | | 7.5 mg: 3-9 kP |
| | Compression | Adjust to meet target | |
| | Force | hardness | |

Film Coating

Coating Suspension Preparation

Purified water was dispensed into a suitably sized volumetric tank and the mixer was started. The 9.2% w/w coating premix (Sepifilm™ 4219 salmon film coating agent) was added to the water over about 5 minutes. The resulting mixture was then mixed for 2 hours until evenly dispersed and no lumps were observed. The mixing speed was then reduced and the suspension was allowed to de-aerate.

Polishing Solution Preparation

Purified water was dispensed into a suitably sized volumetric tank. The mixer was started and the 10.0% w/w polyethylene glycol 6000 was added to the water and the resulting mixture was mixed until completely dissolved.

Film Coating

The core tablets prepared at the end of the compression were loaded into a coating pan. The tablet bed was then heated until an exhaust temperature of 43° C. was achieved. Once the exhaust temperature was stable, the Sepifilm™ 4219 salmon film coating suspension was sprayed until the target mass of the suspension had been applied. The line from the film coating suspension was changed to the polyethylene glycol 6000 polishing solution and the lines were purged. While this was being undertaken, the exhaust temperature was maintained at 43° C. The polyethylene glycol 6000 polishing solution was then sprayed onto the tablets. Once this was complete, the tablets were cooled to less than 30° C. and then discharged into high density polyethylene drums, double lined with 2 polyethylene bags. A desiccant pouch was placed between the inner and outer bags. The control strategy for the film coating is shown in Table 8.

TABLE 8
Ivabradine Film-Coating Strategy

| | | Parameter Target | |
|---|---|---|---|
| Unit Operation | In process Control | Set-Point | Material Attribute |
| Suspension | Mixing speed | Adjust for vortex | De-aerate prior to |
| preparation | Sepifilm ™ 4219 Addition | Add over 5 minutes | spraying (variable |
| | rate | | time) |
| | Mixing Time | 2 hours | |
| Solution | Mixing speed | Adjust for vortex | N/A |
| preparation | Mixing time | Stir until dissolved | |
| Film Coating | Inlet air flow | 2600 CFM | N/A |
| | Exhaust air temperature | 43° C. | |
| | Pan Speed | 6 RPM | |
| | Suspension spray rate | 600 g/min | |
| | Atomizing and pattern air | Adjust for spray pattern | |
| Polishing | Inlet air flow | 2600 CFM | N/A |
| | Exhaust air temperature | 43° C. | |
| | Pan Speed | 6 RPM | |
| | Solution spray rate | 600 g/min | |
| | Atomizing and pattern air | Adjust for spray pattern | |
| Cocling | Inlet air flow | Off | Exhaust |
| | | | temperature ≤ 30° C. |
| | Exhaust air temperature | Measured | AQL inspection |
| | Pan speed | Jog | Final tablet release |
| | | | testing |

All publications, patents, and patent applications cited in this specification are hereby incorporated by reference herein in their entireties and for all purposes as if each individual publication or patent application were specifically and individually indicated as being incorporated by reference and as if each reference was fully set forth in its entirety. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1. A method of preparing an ivabradine hydrochloride granule product, the method comprising:
a) mixing lactose monohydrate, maize starch, maltodextrin, ivabradine hydrochloride, and water to provide a wet granule product, wherein
the percent of lactose monohydrate by weight used to make the wet granule product ranges from 55% to 65% based on the total combined weight of the lactose monohydrate, maize starch, maltodextrin, ivabradine hydrochloride, and water;
the percent of maize starch by weight used to make the wet granule product ranges from 17% to 22% based on the total combined weight of the lactose monohydrate, maize starch, maltodextrin, ivabradine hydrochloride, and water;
the percent of maltodextrin by weight used to make the wet granule product ranges from 8% to 11% based on the total combined weight of the lactose monohydrate, maize starch, maltodextrin, ivabradine hydrochloride, and water;
the percent of ivabradine hydrochloride by weight used to make the wet granule product ranges from 4% to 8% based on the total combined weight of the lactose monohydrate, maize starch, maltodextrin, ivabradine hydrochloride, and water; and
the percent of water by weight used to make the wet granule product ranges from 5% to 8% based on the total combined weight of the lactose monohydrate, maize starch, maltodextrin, ivabradine hydrochloride, and water; and
b) drying the wet granule product to a level of less than or equal to 4% loss on drying to provide the ivabradine hydrochloride granule product,
wherein the wet granule product is dried in a fluid bed dryer with an inlet air temperature ranging from 37° C. to 44° C., with an inlet air dewpoint ranging from 7° C. to 12° C., and with an airflow ranging from 300 to 600 cubic feet per minute.

2. The method of claim 1, wherein
the percent of lactose monohydrate by weight used to make the wet granule product ranges from 59% to 62% based on the total combined weight of the lactose monohydrate, maize starch, maltodextrin, ivabradine hydrochloride, and water;
the percent of maize starch by weight used to make the wet granule product ranges from 18% to 20% based on the total combined weight of the lactose monohydrate, maize starch, maltodextrin, ivabradine hydrochloride, and water;
the percent of maltodextrin by weight used to make the wet granule product ranges from 9% to 11% based on the total combined weight of the lactose monohydrate, maize starch, maltodextrin, ivabradine hydrochloride, and water;
the percent of ivabradine hydrochloride by weight used to make the wet granule product ranges from 5% to 6% based on the total combined weight of the lactose monohydrate, maize starch, maltodextrin, ivabradine hydrochloride, and water; and
the percent of water by weight used to make the wet granule product ranges from 5.2% to 6.5% based on the total combined weight of the lactose monohydrate, maize starch, maltodextrin, ivabradine hydrochloride, and water.

3. The method of claim 1, wherein
the percent of lactose monohydrate by weight used to make the wet granule product ranges from 60% to 62% based on the total combined weight of the lactose monohydrate, maize starch, maltodextrin, ivabradine hydrochloride, and water;
the percent of maize starch by weight used to make the wet granule product ranges from 19% to 20% based on the total combined weight of the lactose monohydrate, maize starch, maltodextrin, ivabradine hydrochloride, and water;
the percent of maltodextrin by weight used to make the wet granule product ranges from 9% to 10% based on the total combined weight of the lactose monohydrate, maize starch, maltodextrin, ivabradine hydrochloride, and water;
the percent of ivabradine hydrochloride by weight used to make the wet granule product ranges from 5% to 5.5% based on the total combined weight of the lactose monohydrate, maize starch, maltodextrin, ivabradine hydrochloride, and water; and
the percent of water by weight used to make the wet granule product ranges from 5.2% to 5.5% based on the total combined weight of the lactose monohydrate, maize starch, maltodextrin, ivabradine hydrochloride, and water.

4. The method of claim 1, wherein
the percent of lactose monohydrate by weight used to make the wet granule product ranges from 60.9% to 61.0% based on the total combined weight of the lactose monohydrate, maize starch, maltodextrin, ivabradine hydrochloride, and water;
the percent of maize starch by weight used to make the wet granule product ranges from 19.05% to 19.1% based on the total combined weight of the lactose monohydrate, maize starch, maltodextrin, ivabradine hydrochloride, and water;
the percent of maltodextrin by weight used to make the wet granule product ranges from 9.5% to 9.6% based on the total combined weight of the lactose monohydrate, maize starch, maltodextrin, ivabradine hydrochloride, and water;
the percent of ivabradine hydrochloride by weight used to make the wet granule product ranges from 5.1% to 5.2% based on the total combined weight of the lactose monohydrate, maize starch, maltodextrin, ivabradine hydrochloride, and water; and
the percent of water by weight used to make the wet granule product ranges from 5.2% to 5.4% based on the total combined weight of the lactose monohydrate, maize starch, maltodextrin, ivabradine hydrochloride, and water.

5. The method of claim 1, wherein
the amount of lactose monohydrate used to make the wet granule product ranges from 63 kg to 65 kg;
the amount of maize starch used to make the wet granule product ranges from 19 kg to 21 kg;
the amount of maltodextrin used to make the wet granule product ranges from 9.5 kg to 10.5 kg;
the amount of ivabradine hydrochloride used to make the wet granule product ranges from 5.2 kg to 5.5 kg; and
the amount of water used to make the wet granule product ranges from 5.3 kg to 6.0 kg.

6. The method of claim 1, wherein
the amount of lactose monohydrate used to make the wet granule product ranges from 63.8 kg to 64 kg;
the amount of maize starch used to make the wet granule product ranges from 19.5 kg to 20.5 kg;
the amount of maltodextrin used to make the wet granule product ranges from 9.8 kg to 10.2 kg;
the amount of ivabradine hydrochloride used to make the wet granule product ranges from 5.3 kg to 5.45 kg; and
the amount of water used to make the wet granule product ranges from 5.4 kg to 5.8 kg.

7. The method of claim 1, wherein
the amount of lactose monohydrate used to make the wet granule product ranges from 63.9 kg to 63.92 kg;
the amount of maize starch used to make the wet granule product ranges from 19.9 kg to 20.1 kg;
the amount of maltodextrin used to make the wet granule product ranges from 9.9 kg to 10.1 kg;
the amount of ivabradine hydrochloride used to make the wet granule product ranges from 5.39 kg to 5.41 kg; and
the amount of water used to make the wet granule product ranges from 5.48 kg to 5.52 kg.

8. The method of claim 1, wherein
the amount of lactose monohydrate used to make the wet granule product is 63.91 kg;
the amount of maize starch used to make the wet granule product is 20.0 kg;
the amount of maltodextrin used to make the wet granule product is 10.0 kg;
the amount of ivabradine hydrochloride used to make the wet granule product is 5.39 kg; and
the amount of water used to make the wet granule product is 5.5 kg.

9. The method of claim 1, wherein
the percent of lactose monohydrate by weight used to make the wet granule product ranges from 56% to 59% based on the total combined weight of the lactose monohydrate, maize starch, maltodextrin, ivabradine hydrochloride, and water;
the percent of maize starch by weight used to make the wet granule product ranges from 17% to 20% based on the total combined weight of the lactose monohydrate, maize starch, maltodextrin, ivabradine hydrochloride, and water;
the percent of maltodextrin by weight used to make the wet granule product ranges from 9% to 11% based on the total combined weight of the lactose monohydrate, maize starch, maltodextrin, ivabradine hydrochloride, and water;
the percent of ivabradine hydrochloride by weight used to make the wet granule product ranges from 7% to 8% based on the total combined weight of the lactose monohydrate, maize starch, maltodextrin, ivabradine hydrochloride, and water; and
the percent of water by weight used to make the wet granule product ranges from 6% to 8% based on the total combined weight of the lactose monohydrate, maize starch, maltodextrin, ivabradine hydrochloride, and water.

10. The method of claim 1, wherein
the percent of lactose monohydrate by weight used to make the wet granule product ranges from 57% to 59% based on the total combined weight of the lactose monohydrate, maize starch, maltodextrin, ivabradine hydrochloride, and water;
the percent of maize starch by weight used to make the wet granule product ranges from 18% to 20% based on the total combined weight of the lactose monohydrate, maize starch, maltodextrin, ivabradine hydrochloride, and water;
the percent of maltodextrin by weight used to make the wet granule product ranges from 9% to 10% based on the total combined weight of the lactose monohydrate, maize starch, maltodextrin, ivabradine hydrochloride, and water;
the percent of ivabradine hydrochloride by weight used to make the wet granule product ranges from 7% to 8% based on the total combined weight of the lactose monohydrate, maize starch, maltodextrin, ivabradine hydrochloride, and water; and
the percent of water by weight used to make the wet granule product ranges from 6% to 7% based on the total combined weight of the lactose monohydrate, maize starch, maltodextrin, ivabradine hydrochloride, and water.

11. The method of claim 1, wherein
the percent of lactose monohydrate by weight used to make the wet granule product ranges from 57.5% to 58% based on the total combined weight of the lactose monohydrate, maize starch, maltodextrin, ivabradine hydrochloride, and water;
the percent of maize starch by weight used to make the wet granule product ranges from 18.5% to 19% based on the total combined weight of the lactose monohydrate, maize starch, maltodextrin, ivabradine hydrochloride, and water;
the percent of maltodextrin by weight used to make the wet granule product ranges from 9.3% to 9.6% based on the total combined weight of the lactose monohydrate, maize starch, maltodextrin, ivabradine hydrochloride, and water;
the percent of ivabradine hydrochloride by weight used to make the wet granule product ranges from 7.5% to 7.8% based on the total combined weight of the lactose monohydrate, maize starch, maltodextrin, ivabradine hydrochloride, and water; and
the percent of water by weight used to make the wet granule product ranges from 6% to 6.5% based on the total combined weight of the lactose monohydrate, maize starch, maltodextrin, ivabradine hydrochloride, and water.

12. The method of claim 1, wherein
the amount of lactose monohydrate used to make the wet granule product ranges from 60 kg to 63 kg;
the amount of maize starch used to make the wet granule product ranges from 19 kg to 21 kg;
the amount of maltodextrin used to make the wet granule product ranges from 9.5 kg to 10.5 kg;
the amount of ivabradine hydrochloride used to make the wet granule product ranges from 7.5 kg to 8.5 kg; and
the amount of water used to make the wet granule product ranges from 6.0 kg to 6.9 kg.

13. The method of claim 1, wherein
the amount of lactose monohydrate used to make the wet granule product ranges from 60.8 kg to 62 kg;
the amount of maize starch used to make the wet granule product ranges from 19.5 kg to 20.5 kg;
the amount of maltodextrin used to make the wet granule product ranges from 9.8 kg to 10.4 kg;
the amount of ivabradine hydrochloride used to make the wet granule product ranges from 7.8 kg to 8.2 kg; and
the amount of water used to make the wet granule product ranges from 6.2 kg to 6.7 kg.

14. The method of claim 1, wherein
the amount of lactose monohydrate used to make the wet granule product ranges from 61.1 kg to 61.3 kg;
the amount of maize starch used to make the wet granule product ranges from 19.9 kg to 20.1 kg;
the amount of maltodextrin used to make the wet granule product ranges from 9.9 kg to 10.1 kg;
the amount of ivabradine hydrochloride used to make the wet granule product ranges from 8.0 kg to 8.2 kg; and
the amount of water used to make the wet granule product ranges from 6.4 kg to 6.6 kg.

15. The method of claim 1, wherein
the amount of lactose monohydrate used to make the wet granule is 61.22 kg;
the amount of maize starch used to make the wet granule product is 20.0 kg;
the amount of maltodextrin used to make the wet granule product is 10.0 kg;
the amount of ivabradine hydrochloride used to make the wet granule product is 8.9 kg; and
the amount of water used to make the wet granule product is 6.5 kg.

16. The method of claim 1, wherein the wet granule product is dried in a fluid bed dryer with an inlet air temperature ranging from 39° C. to 43.2° C., with an inlet air dewpoint ranging from 9° C. to 11° C., and with an airflow ranging from 450 to 550 cubic feet per minute.

17. The method of claim 1, wherein the wet granule product is dried in a fluid bed dryer with an inlet air temperature ranging from 40° C. to 43° C., with an inlet air dewpoint ranging from 9.5° C. to 10.5° C., and with an airflow ranging from 480 to 520 cubic feet per minute.

18. The method of claim 1, wherein the wet granule product is dried in a fluid bed dryer with an inlet air temperature ranging from 40° C. to 43° C., with an inlet air dewpoint of 10° C., and with an airflow of 500 cubic feet per minute.

19. The method of claim 1, wherein the method further comprises
c) milling the wet granule product prior to drying the wet granule product.

20. The method of claim 19, wherein the milling of the wet granule product is conducted while maintaining the dew point at a range from 8° C. to 15° C., and maintaining an air inlet temperature of 8° C. to 15°.

21. The method of claim 19, wherein the the milling of the wet granule product is conducted while maintaining the dew point at at a range from 9° C. to 11° C., and maintaining an air inlet temperature of 9° C. to 11° C.

22. The method of claim 19, wherein the the milling of the wet granule product is conducted while maintaining the dew point at 10° C., and maintaining an air inlet temperature of 10° C.

23. A method of making ivabradine hydrochloride precompression product, the method comprising:
a) milling the ivabradine hydrochloride granule product produced using the method of claim 1 to form a milled ivabradine granule product; and
b) mixing the milled ivabradine granule product with magnesium stearate and colloidal silica to produce the ivabradine hydrochloride precompression product, wherein
the percent of magnesium stearate by weight used to make the ivabradine hydrochloride precompression product ranges from 0.4% to 0.6% based on the total combined weight of the lactose monohydrate, maize starch, maltodextrin, ivabradine hydrochloride, magnesium stearate, and colloidal silica in the ivabradine hydrochloride precompression product; and
the percent of colloidal silica by weight used to make the ivabradine hydrochloride precompression product ranges from 0.1% to 0.3% based on the total combined weight of the lactose monohydrate, maize starch, maltodextrin, ivabradine hydrochloride, magnesium stearate, and colloidal silica in the ivabradine hydrochloride precompression product.

24. The method of claim 23, wherein
the percent of magnesium stearate by weight used to make the ivabradine hydrochloride precompression product ranges from 0.45% to 0.55% based on the total combined weight of the lactose monohydrate, maize starch, maltodextrin, ivabradine hydrochloride, magnesium stearate, and colloidal silica in the ivabradine hydrochloride precompression product; and
the percent of colloidal silica by weight used to make the ivabradine hydrochloride precompression product ranges from 0.15% to 0.25% based on the total combined weight of the lactose monohydrate, maize starch, maltodextrin, ivabradine hydrochloride, magnesium stearate, and colloidal silica in the ivabradine hydrochloride precompression product.

25. The method of claim 23, wherein
the percent of magnesium stearate by weight used to make the ivabradine hydrochloride precompression product ranges from 0.48% to 0.52% based on the total combined weight of the lactose monohydrate, maize starch, maltodextrin, ivabradine hydrochloride, magnesium stearate, and colloidal silica in the ivabradine hydrochloride precompression product;
the percent of colloidal silica by weight used to make the ivabradine hydrochloride precompression product ranges from 0.18% to 0.22% based on the total combined weight of the lactose monohydrate, maize starch, maltodextrin, ivabradine hydrochloride, magnesium stearate, and colloidal silica in the ivabradine hydrochloride precompression product.

26. The method of claim 23, wherein
the amount of magnesium stearate used to make the ivabradine hydrochloride precompression product ranges from 0.48 kg to 0.52 kg; and
the amount of colloidal silica used to make the ivabradine hydrochloride precompression product ranges from 0.18 kg to 0.22 kg.

27. The method of claim 23, wherein
the amount of magnesium stearate used to make the ivabradine hydrochloride precompression product is 0.50 kg; and
the amount of colloidal silica used to make the ivabradine hydrochloride precompression product is 0.20 kg.

28. A method for making precoated tablets of ivabradine hydrochloride, the method comprising: compressing the ivabradine hydrochloride precompression product of claim 23 to produce the precoated tablets of ivabradine hydrochloride.

29. The method of claim 28, wherein the 100 mg of the ivabradine hydrochloride precompression product is compressed to form the precoated tablets of ivabradine hydrochloride.

30. A method of forming ivabradine hydrochloride drug product, the method comprising:
a) coating the precoated tablets of ivabradine hydrochloride of claim 28 with an aqueous suspension or solution comprising a coloring agent to form a color-coated ivabradine hydrochloride tablet; and
b) coating the color-coated ivabradine hydrochloride tablet with an aqueous solution of a polishing agent.

31. The method of claim 30, wherein the coloring agent is Sepifilm™ salmon film-coating agent.

32. The method of claim 30, wherein the polishing agent is polyethylene glycol 6000.
